(19) United States
(12) Patent Application Publication
(10) Pub. No.: US 2008/0260879 A1
(43) Pub. Date: Oct. 23, 2008

(54) Use of Paeonia and Extracts Thereof with Growth Promoting Activity in Veterinary Medicine and Zootechnics

(76) Inventors: Andrea Giori, Milano (IT); Gianpaolo Forni, Milano (IT); Alessandro Anelli, Milano (IT)

Correspondence Address: YOUNG & THOMPSON, 209 Madison Street, Suite 500, ALEXANDRIA, VA, 22314, US

(21) Appl. No.: 11/659,332

(22) PCT Filed: Jul. 20, 2005
(86) PCT No.: PCT/EP05/07895

§ 371 Date: Apr. 6, 2007

(30) Foreign Application Priority Data
Aug. 6, 2004 (IT) MI2004A001630

(51) Int. Cl. A61K 36/71

(52) U.S. Cl. 424/773; 424/725

(57) ABSTRACT

The present invention relates to the use of extracts of plants or roots of the Paeonia genus, in particular Paeonia lactiflora, for the preparation of growth promoters for use in livestock.

The present invention relates to the use of extracts of plants or roots of the genus Paeonia, in particular Paeonia lactiflora, for the preparation of growth promoters for use in livestock.

### TECHNOLOGICAL BACKGROUND

Bacterial antibiotic resistance is an increasing problem which affects public health, including the veterinary field.

The use in veterinary medicine of antibiotics that are mostly the same as or strictly related to those used in human medicine, and the established practice of use of antibiotics at sub-therapeutical doses as growth promoters and mass metaphylactic agents, are the main causes of diffusion of acquired antibiotic resistance by a number of bacterial species responsible for pathologies in domestic animals and often even in humans. The presence of antibiotic residues in meat and, indirectly, the selection of antibiotic-resistant bacteria, which can transfer the resistance genes to bacteria pathogenic to humans, can be a hazard to human health.

Approximately 25% of the total antibiotic production is intended for use in livestock, the 90% of them being used at sub-therapeutical doses for both the prevention of diseases and the promotion of growth. Antimicrobials have been added to feed and extensively used in zootechnics since the '50s to improve productive performances. In swine breeding, for instance, weaning of piglets is the most delicate step, as the distressing events related to the transfer and mixing of different groups of animals and the changes of feed induce alterations in the intestinal microbial flora of the animal, making it more susceptible to infections. The use of antibiotics in the prophylaxis of endemic diseases occurring during weaning has lead to a generalized use of antibiotics which are included in feed for times which can be protracted until the animal is even 70-80 day old.

As a consequence of antibiotic-resistance emergency, the European Union has followed a restricted approach concerning the use of antibiotics as growth promoters, antimicrobial prophylaxis, multi-therapies, mass medication through feed and water. In particular, EU is withdrawing all authorizations concerning the use of antibiotics as growth promoters by 2006. However, the prohibition of use of antibiotics as growth promoters and prophylactic agents will involve a deterioration of animals health, with an increase in diarrhoea, weight loss and mortality.

Different approaches exist for reducing the dependency of zootechnics, more particularly swine breeding, on the use of antibiotics: the choice one is the development of alternatives acting through the control of bacteria and promoting the natural reactivity of the animal, thereby inducing an improvement in health as well as the optimization of performances. Mandatory characteristics for such alternative solutions are safety of use and cheapness for producers, and hence for consumers.

Paeonia belongs to the Paeoniaceae family. The Paeonia genus, although including a comparatively reduced number of species, is taxonomically complex. The roots of plants of the genus Paeonia have been used for a long time, as they are or in the form of aqueous or water-ethanol extracts, in Chinese traditional medicine for the treatment of a variety of conditions including hypertension, muscle spasms, fever, diseases of the female reproductive system, haemorrhage.

In particular Paeonia lactiflora, original from Siberia, Mongolia and northern China, produces more than one flower per stem. It was introduced in Western countries in the 19^{th} century, and it gave rise to a large number of cultivars and hybrids. The active principles present in Paeonia lactiflora include the glycoside paeoniflorin as well as proanthocyanidins, flavonoids, tannins, polysaccharides.

### DISCLOSURE OF THE INVENTION

It has now been found that the administration of parts of plants, particularly roots, or extracts of plants of the Paeonia genus, in particular Paeonia lactiflora, to livestock, is at least as much as active as the administration of growth-promoting antibiotics.

Therefore, the present invention relates to compositions for the veterinary or zootechnical use, comprising as active ingredients ground roots or extracts of roots of Paeonia, preferably of Paeonia lactiflora, admixed with suitable carriers or in the form of feed supplements.

Said compositions proved capable of promoting growth in livestock, in particular swine, bovine, ovine and equine, more particularly swine, without inducing the drawbacks and side effects of growth-promoting antibiotics.

Dosages will depend on a number of factors, such as species, size, weight and age of the animal. In principle, a typical dosage will range from 0.1 g to 10 g of extract/root per animal/day.

The extracts usable according to the invention can be prepared by a process which comprises:
a) extraction of Paeonia lactiflora roots with a solvent (native extract), and optionally
b) purification from water insolubles.

Step (a) of extraction of Paeonia lactiflora roots involves the use of water or a C1-C3 alcohol, or mixtures thereof, or aqueous solutions thereof. A water-ethanol solution with 90% content alcohol (v/v) is preferred, but an alcoholic solution with water content from 5% v/v to 100% v/v can also be used. The extraction temperature can range from 5° C. to the boiling temperature of the selected solvent, preferably 70° C.

The native extract can be purified by removing water insolubles (step b).

The native extract from step (a) is concentrated under reduced pressure at 60° C., completely removing ethanol (or any other solvents used among those cited above) from the solution, then the extract is concentrated to a dry residue 5% w/w to 55% w/w, preferably 25% w/w, and kept at temperatures of 1° C. to 30° C., preferably 4° C., without stirring for a time from 1 hour to 32 hours, preferably 16 hours.

The resulting clear solution, consisting of the purified extract, is separated from insolubles by centrifugation.

The resulting extracts, having a content in paeoniflorin and albiflorin ranging from 5 to 30%, are a further aspect of the invention.

The following examples illustrate the invention in further detail.

### EXAMPLE 1

### Preparation of a Paeonia lactiflora Roots Extract for the Zootechnical Use

Step a): Extraction of Paeonia lactiflora Roots with a Hydro-Alcoholic Solution

1000 grams of Paeonia lactiflora roots are dried and finely ground, then extracted with 0.8 litres of 90% v/v ethanol at 70° C. for 4 hours in a jacketed static percolator. After 4 hours, the percolate is recovered and extraction is repeated for a further 7-8 times under the same conditions. The combined percolates are hot filtered with suction and concentrated by a rotary evaporator at 60° C. under reduced pressure.

The extract is further dried under vacuum at 60° C. for 24 hours to obtain a yellow, easy-to-grind powder. 322 g of product are obtained, corresponding to a 32.2% w/w weight yield, having HPLC paeoniflorin content of 9.62% w/w, albilforin content of 1.26% w/w, for a total value of 10.88% w/w.

Step b): Purification of the Paeonia lactiflora Roots Native Extract.

The native extract obtained in step a), having a residue of 322 g, is concentrated under reduced pressure at 60° C. thus removing all of the solvent except water. The resulting aqueous suspension is concentrated to 25% w/w dry residue and left to stand for 16 hours without stirring at 4° C.

After 16 hours at 4° C., the aqueous suspension is centrifuged to remove insolubles from the solution, which is dried at 60° C. under vacuum to obtain 231.4 g of a dry purified extract having HPLC paeoniflorin content of 13.41% w/w and albilforin content of 1.74% w/w, for a total value of the purified extract of 15.15% w/w by HPLC.

The weight yield of the purified extract to the starting material is 23.1% w/w.

### EXAMPLE 2

### In Vivo Test

Experiments were carried out on 120 piglets divided into 4 groups of 30 animals each.

Group 1 (control) was treated with standard feed free from additives. Group 2 (antibiotic) was treated with medicated feed containing 2 g of apramycin and 1 g of colistin per kg of feed. Group 3 was treated with standard feed added with 2 g/Kg of Paeonia extract. Group 4 was treated with standard feed added with 5 g/kg of finely ground Paeonia roots.

All animals were treated during weaning from day 22 to day 41.

The animal well being was evaluated by the total weight increase, the feed daily intake and the percentage of diarrhoeic animals. Moreover, from day 41, faeces samples were microbiologically analysed for bacterial counts of total mesophyte charge, total Coliform and Lactobacilli. All parameters were evaluated with standard methods known to those skilled in the art.

Results reported in the following Tables prove that the addition to standard feed of Paeonia in the form of both finely ground roots and water-ethanol extract, although being devoid of per se in vitro antibacterial activity, can be a valuable replacement for antibiotic growth promoters in weaning foodstock,

TABLE 1
Piglets weights and Mean Weight Increase (MWI) on day 41

Piglets weight (kg)

| Treatment | day 22 | day 41 | MWI |
|---|---|---|---|
| Control | 7.02 ± 1.2 | 10.25 ± 1.5 | 3.22 |
| Antibiotic | 7.43 ± 1.3 | 12.21 ± 1.6 | 4.78 |
| Paeonia roots | 7.29 ± 1.2 | 11.49 ± 1.4 | 4.20 |
| Paeonia water- | 7.45 ± 1.3 | 11.96 ± 1.3 | 4.51 |
| ethanol extract | | | |

TABLE 2
Mean daily intake: amount of feed daily intake, expressed in
kg, between days 22 and 41 of treatment

| Treatment | kg |
|---|---|
| Control | 0.22 |
| Antibiotic | 0.37 |
| Paeonia roots | 0.31 |
| Paeonia water-ethanol extract | 0.35 |

TABLE 3
Effect of treatment on diarrhoea onset in the various treated
groups.

| | % of animals |
|---|---|
| Treatment | with diarrhoea |
| Control | 60 |
| Antibiotic | 10 |
| Paeonia roots | 30 |
| Paeonia water-ethanol extract | 20 |

TABLE 4
Effect of administration of the tested substances on different
bacterial populations (log Ufc/g) from faeces samples taken on day 41

| Treatment | E. Coli | CBT | Lactobacilli |
|---|---|---|---|
| Antibiotic | 6.41 | 6.47 | 5.38 |
| Control | 8.05 | 7.55 | 6.93 |
| Paeonia roots | 6.88 | 7.09 | 6.96 |
| Paeonia water- | 6.65 | 6.79 | 6.90 |
| ethanol extract | | | |

1. A method of promoting growth in an animal comprising administering an effective amount of parts or extracts of plants of the Paeonia genus to an animal in need thereof.

2. The method according to claim 1, wherein the plant of the Paeonia genus is Paeonia lactiflora.

3. The method according to claim 1, wherein the parts of Paeonia are the roots.

4. The method according to claim 1, wherein the extract is a roots hydro-alcoholic extract.

5. Veterinary or zootechnical compositions comprising parts or extracts of plants of the Paeonia genus in admixture with suitable carriers.

6. Compositions as claimed in claim 5 comprising roots or hydro-alcoholic extracts of Paeonia lactiflora roots.

7. Feed comprising roots or root extracts of plants of the Paeonia genus, in particular Paeonia lactiflora.

8. Paeonia lactiflora extracts comprising paeoniflorin and albiflorin content ranging from 5 to 30%.

9. The method according to claim 2, wherein the parts of Paeonia are the roots.

10. The method according to claim 2, wherein the extract is a roots hydro-alcoholic extract.

11. The method according to claim 1, wherein the parts or extracts of the plants of the Paeonia genus are in admixture with suitable carriers.

12. The method according to claim 1, wherein the effective amount is 0.1 g to 10 g of extract/root per animal/day.
